(19) United States
(12) Patent Application Publication
(10) Pub. No.: US 2008/0233128 A1
(43) Pub. Date: Sep. 25, 2008

(54) Treatment of Viral Infections

(76) Inventor: Werner Krause, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/933,691

(22) Filed: Nov. 1, 2007

Related U.S. Application Data
(60) Provisional application No. 60/856,044, filed on Nov. 2, 2006.

(51) Int. Cl. A61K 39/42; A61P 31/12

(52) U.S. Cl. 424/147.1

(57) ABSTRACT

A method of treating viral infections comprises administering to a patient a regimen that is able to temporarily reduce the number or functionality of the host cells which the virus uses for its reproduction in a controlled manner. Preferably, host cells are part of the immune system.

This application claims the benefit of the filing date of U.S. Provisional Application Ser. No. 60/856,044, filed Nov. 2, 2006, which is incorporated by reference herein.

### FIELD OF THE INVENTION

The present invention relates to the treatment of patients with viral infections. The invention involves temporarily reducing the number or functionality of cells (host cells) that are used by the virus for its reproduction. Most preferably, host cells of the immune system are covered by this invention. In killing the host cells, the virus is killed together with the infected host cells and potentially surviving, circulating viruses are prevented from reproduction. “Conventional” anti-virus therapy might be added to this regimen in order to eliminate remaining viruses.

### BACKGROUND OF THE INVENTION

Viral infections still represent a great threat to the health and well-being of many persons, particularly in third-world countries with low standards of hygiene. After having entered the human organism, a virus cannot reproduce on its own, i.e. without the assistance of host cells. The virus will enter these cells, introduce its own RNA or DNA into the nucleus and induce the production of replicates thereof. Likewise, any proteins and other building blocks for a new virus are produced by the host cell. Finally, the newly created viruses are released from the host cell, which normally is either temporarily or continuously reduced in its functionality or even destroyed. There is a plethora of potential host cells available in the organism and different viral types are specialized on different kinds of host cells. The influenza virus, for example, is using the cells lining the respiratory tract for reproduction, the virus causing poliomyelitis is using nerve cells and viral hepatitis is originating from the infection of liver cells.

The particular virus, HIV, like other viruses, cannot reproduce without the aid of a living cell. Although HIV can infect a number of cells in the body, its main target is T-cells, or more specifically, CD4 helper cells. T-cells are an important part of the immune system because they help facilitate the body's response to many common but potentially fatal infections. Without enough T-cells, the body's immune system is unable to defend itself against many infections. HIV's life cycle directly causes a reduction in the number of T-cells in the body, eventually resulting in an increased risk of infections.

After HIV enters the body, it comes in contact with its preferred host cell—the T-cell. HIV will take over the host cell's cellular machinery to reproduce thousands of copies of itself. HIV has to complete many steps in order for this to happen. At each step of HIV's life cycle, it is theoretically possible to design a drug that will stop the virus. The individual steps of the virus's reproduction process are the basis for all currently available drugs that fight HIV infection. In addition, treatments try to reconstitute the body's immune system that is compromised and finally destroyed by HIV or improve it by co-administered drugs.

As is known (see, e.g., ACRIA Update 12(1), 2002/3), once HIV comes into contact with a T-cell, it must attach itself to the cell so that it can fuse with the cell and inject its genetic material into it. Attachment means specific binding between proteins on the surface of the virus and receptors on the surface of the T-cell. Normally, these receptors help the cell communicate with other cells. Two receptors in particular, CD4 and a beta-chemokine receptor (either CCR5 or CXCR4), are used by HIV to latch onto the cell. On the surface of the viral envelope, two sets of proteins (antireceptors) called gp120 and gp41 attach to CD4 and CCR5/CXCR4.

Attachment or entry inhibitors are currently being studied in clinical trials. These drugs block the interaction between the cellular receptors and the antireceptor on the virus by binding to or altering the receptor sites. People who naturally lack these cellular receptors because of a genetic mutation, or those who have them blocked by natural chemokines (chemical messengers), may not get infected as readily with HIV or may progress more slowly to AIDS. Currently also vaccines are being examined that may help the body block these receptors.

After attachment is completed, viral penetration occurs. Penetration allows the nucleocapsid of the virus to be injected directly into the cell's cytoplasm. gp120 actually contains three glycosylated proteins (glycoproteins) and, once gp120 attaches itself to CD4, these three proteins spread apart. This allows the gp41 protein, which is normally hidden by the gp120 proteins, to become exposed and bind to the chemokine receptor. Once this has occurred, the viral envelope and the cell membrane are brought into direct contact and essentially fuse into each other.

Fusion inhibitors prevent the binding of gp41 and the chemokine receptor. T-20 (enfuvirtide, Fuzeon) binds to a portion of gp41, preventing it from binding to the chemokine receptor.

Once HIV has penetrated the cell membrane, it is ready to release its genetic information (RNA) into the cell. The viral RNA is contained in the nucleocapsid. The nucleocapsid needs to be partially dissolved so that the virus's RNA can be converted into DNA, a necessary step if HIV's genetic material is to be incorporated into the T-cell's genetic core.

HIV's RNA is converted to DNA by reverse transcription. HIV uses reverse transcriptase to accomplish this transcription. The single-stranded viral RNA is transcribed into a double strand of DNA, which contains the instructions HIV needs to take over a T-cell's genetic machinery in order to reproduce itself. Reverse transcriptase uses nucleotides from the cell cytoplasm to make this process possible.

Reverse transcriptase inhibitors block HIV's reverse transcriptase from using these nucleotides. Nucleoside and nucleotide analog reverse transcriptase inhibitors (NRTIs)—such as Zerit, Epivir, and Viread—contain faulty imitations of the nucleotides found in a T-cell's cytoplasm. Instead of incorporating a nucleotide into the growing chain of DNA, the imitation building blocks in NRTIs are inserted, which prevents the double strand of DNA from becoming fully formed. Non-nucleoside reverse transcriptase inhibitors (NNRTIs)—such as Viramune and Sustiva—block reverse transcription by attaching to the enzyme in a way that prevents it from functioning.

If HIV succeeds in transforming its instructions from RNA to DNA, HIV must then insert its DNA (the pre-integration complex) into the cell's DNA. This process is called integration. In most human cells, DNA is stored in the cell nucleus. In order for integration to occur, the newly formed DNA must be transported across the nuclear membrane into the nucleus.

Although the exact mechanism that HIV uses to transport its genetic material into the cell nucleus is still unclear, viral protein R (VPR), which is carried by HIV, may facilitate the movement of the pre-integration complex to the nucleus. Once the viral RNA has successfully bridged the nuclear membrane and been escorted to the nucleus, HIV uses the enzyme integrase to insert its double-stranded DNA into the cell's existing DNA.

Drugs that inhibit the HIV pre-integration complex from traveling to the nucleus—integrase inhibitors—are currently in clinical trials.

After successful integration of the viral DNA, the host cell is now latently infected with HIV. This viral DNA is referred to as provirus. The HIV provirus now awaits activation. When the immune cell becomes activated, this latent provirus awakens and instructs the cellular machinery to produce the necessary components of HIV. From the viral DNA, two strands of RNA are constructed and transported out of the nucleus. One strand is translated into subunits of HIV such as protease, reverse transcriptase, integrase, and structural proteins. The other strand becomes the genetic material for the new viruses. Compounds that inhibit or alter viral RNA have been identified as potential antiviral agents.

Once the various viral subunits have been produced and processed, they must be separated for the final assembly into new virus. This separation, or cleavage, is accomplished by the viral protease enzyme.

Protease inhibitors—such as Kaletra, Crixivan, and Viracept—bind to the protease enzyme and prevent it from separating, or cleaving, the subunits.

If cleavage is successfully completed, the HIV subunits combine to make up the content of the new virions. In the next step of the viral life cycle, the structural subunits of HIV mesh with the cell's membrane and begin to deform a section of the membrane. This allows the nucleocapsid to take shape and viral RNA is wound tightly to fit inside the nucleocapsid. Zinc finger inhibitors, which interfere with the packaging of the viral RNA into the nucleocapsid are currently studied as anti-viral drugs.

The final step of the viral life cycle is budding. In this process, the genetic material enclosed in the nucleocapsid merges with the deformed cell membrane to form the new viral envelope. With its genetic material tucked away in its nucleocapsid and a new outer coat made from the host cell's membrane, the newly formed HIV pinches off and enters into circulation, ready to start the whole process again.

During HIV's life cycle, the T-cell, i.e. the host cell for HIV reproduction, is altered and perhaps damaged, causing the death of the cell. It is not exactly known how the cell dies but a number of scenarios have been proposed. First, after the cell becomes infected with a virus, internal signals may tell it to commit suicide. Apoptosis or programmed cell death is a self-destruct program intended to kill the cell with the hopes of killing the virus as well. A second possible mechanism for the death of the cell is that, as thousands of HIV particles bud or escape from the cell, they severely damage the cell's membrane, resulting in the loss of the cell. Another possible cause for the cell's death is that other cells of the immune system, killer cells, recognize that the cell is infected and destroy it.

Whatever the mechanism of the cell's death, there is one less T-cell in the body, and with this happening on a monumental scale, T-cells begin to decline. Over time, there are not enough T-cells to defend the body. At this stage, a person has acquired immunodeficiency syndrome (AIDS), and becomes susceptible to infections that a healthy immune system could deal with. If this process of immune destruction is halted, a weakened immune system may be able to repair some of the damage over time.

As can be seen, the current approaches to treating HIV infection may be summarized as: “fight the virus and improve functioning of the immune system”.

### SUMMARY OF THE INVENTION

This invention involves a shift in paradigm by reducing the number or functionality of host cells—for a certain period of time—in a controlled manner, before this is accomplished by the virus. In case the host cells are killed, any virus located within a host cell would be killed together with the host cell. If the functionality of the host cell is reduced, this would mean that reproduction of the virus is no longer possible. The virus then would either die on its own or would be killed by additional anti-viral therapy.

The invention is applicable not only to humans but also to animals and plants.

As detailed above, in HIV infection, the host cells are T-cells or, more specifically, CD4 helper cells. The present invention would therefore involve in this case shutting down the immune system—for a certain period of time—by killing most or all T-cells or by modifying them using T-cell depletors or T-cell modifiers such that the T-cells are no longer recognized by or available to HIV, thereby saving the immune system from destruction. By doing so the virus cannot use the T-cells for reproduction and, additionally, the virus entrapped in infected T-cells will be killed together with the T-cells. Still circulating viruses, not yet having achieved T-cell infection, will try to enter remaining T-cells if any are left. They will be killed by a second or further courses of treatment. Additional “conventional” anti-HIV treatments, e.g., as described in the paragraphs above, will also contribute to the elimination of HIV and HIV-infected cells. The treatment will be continued until substantially all viruses have been killed. Thereafter, the immune system is allowed to recover.

An advantage of the proposed regimen is that the immune system is not damaged but only shut down. Whereas HIV shuts down the system by simultaneously modifying it such that surviving or newly formed T-cells are no longer “normally” functioning, the shutting down with T-cell depletors does not result in damage of the system and newly formed T-cells—after discontinuation of treatment—are fully functional. However, it will take some time for the normal number of T-cells to reappear. This time depends on the specific drug used for T-cell depletion and on the additional use of immune stimulators such as G-CSF or GM-CSF. The re-establishment of a functioning immune system is not restricted to these two examples (G-CSF or GM-CSF). Any other measures known in the art may be used. During the time of treatment and during the time period of recovery of the immune system, the patients are carefully monitored and treated with anti-bacterial and antiviral drugs in order to prevent other than HIV infections. This prophylaxis is well known to those skilled in the art and constitutes daily life in the treatment of cancer or transplant patients with T-cell depletors (Semin Hematol. 2004 July; 41(3): 224-33, Leuk Lymphoma 2004 April; 45(4): 711-4).

In a special embodiment, this invention therefore relates to a method of treating HIV infection comprising administering to a patient a drug that is able to kill T-cells or modify T-cells such that they are no longer recognized by HIV. The drug may be combined with “conventional” anti-HIV therapy used either as an additional single-drug treatment or given as a drug cocktail.

A good protection against viral infection normally is vaccination. In all cases where vaccination works, the present invention would not necessarily have to be applied, but such use is included. However, if vaccination has not been successful, for example in those cases where the virus attacks the immune system, use of the present invention is indicated.

One of the major threats to human health undoubtedly is HIV infection and AIDS which is caused by a virus that attacks and destroys the immune system. Therefore, the present invention is primarily explained for HIV infection both above and below. Due to the attack on the immune system and the extreme variability in the surface of the virus, vaccination has not been successful in this indication. Using this example, however, does not limit the invention to this disease.

According to the invention, patients with HIV infection are treated with drugs that are able to kill T-cells or to modify the function of T-cells making them no longer recognizable to HIV. Drugs of this kind are for example monoclonal antibodies that bind to specific epitopes on T-cells and effectively kill these cells, such as the CD3 antigen. A drug binding to the T3 antigen is muromonab-CD3 (Orthoclone OKT3). Another potential epitope is the CD52 antigen, which is found on B-cells and T-cells. An example for an antibody binding to the CD52 epitope is alemtuzumab (Campath). However, the invention is not restricted to these types of compounds. Any epitope on T-cells implicated in any way in HIV T-cell attack and, e.g., to which an antibody can be directed, can be utilized, as can any drug that kills T-cells. Moreover, any other type of drug that is able to kill T-cells or prevent them from being recognized by HIV as functioning T-cells, i.e. any T-cell depletor or T-cell function modifier, irrespective of their individual mechanisms of action, may be used. Another example is anti-thymocyte globulin, ATG (Thymoglobulin). Thymoglobulin is anti-thymocyte rabbit immunoglobulin that induces immunosuppression as a result of T-cell depletion and immune modulation. Thymoglobulin is made up of a variety of antibodies that recognize key receptors on T-cells and leads to inactivation and killing of the T-cells. Regarding drugs which modify T-cells, all will be appropriate as long as the result is that the T-cells are no longer recognized by HIV and thus the latter does not invade them. One such exemplary modification is an antibody binding to receptors such as those described above or others, where the binding does not kill T-cells, but does disguise the T-cells so that HIV does not recognize them.

The purpose of intentionally killing T-cells is multifold. For example, any virus in such a T-cell will be killed together with the T-cell. Also, the virus needs T-cells for reproduction. If these are not available, the virus is not able to reproduce. Further, any T-cells or progenitor cells that have survived a reproduction cycle of the virus and subsequently have been damaged or modified by the virus will be killed as well. The objective of doing what looks like the same as the virus is doing, is to do it in a controlled manner and prior to any or serious damage to the system induced by the virus. It is well known from other diseases such as chronic lymphocytic leukemia (CLL) or transplantation of solid organs that after controlled T-cell depletion, the system recovers to its full function. Moreover, it has been clearly established that the time period during which the body is depleted of T-cells can be handled without running an uncontrolled risk for infection. Concomitant antibacterial and antiviral treatment of patients on muromonab-CD3 or alemtuzumab therapy has been established and is well known to those skilled in the art. See, e.g., Tex Heart Inst J. 1988; 15 (2): 102-106. Likewise any other expected side-effects of this type of therapy, such as the cytokine release syndrome, have been well described and can be handled appropriately.

T-cell depletion has been extensively demonstrated for drugs like alemtuzumab or Thymoglobulin. A single dose of alemtuzubmab (Campath) is able to kill all circulating T-cells. This is illustrated in FIG. 1 (Weinblatt et al. Arth & Rheum 38(11):1589-1594, 1995). As can be seen from FIG. 1, full recovery of T-cells takes 3 months or longer. If the treatment is repeated, T-cell count will remain at low levels or zero during a prolonged period of time. With each new dose of alemtuzumab, remaining T-cells will be killed together with any virus having infected the cells. A consecutive treatment course or a series of courses therefore will stepwise reduce the population of HIV cells and finally bringing them to zero. Alemtuzumab is dosed in CLL three times a week at 30 mg for a total of 4-12 consecutive weeks. The final dose of 30 mg is reached after stepwise increases from 3 mg via 10 mg to 30 mg in the first week. In HIV infection, smaller doses will be indicated since the tumor load in CLL takes up most of the drug during administration in the first part of the therapy. In multiple sclerosis (MS), where alemtuzumab is also studied, dosing is restricted to five daily doses of 10-30 mg for one week. In MS, the therapy might be repeated after a full year.

T-cell depletion after Thymoglobulin is illustrated in FIG. 2 (taken from the Thymoglobulin Prescribing Information). Thymoglobulin is infused in GVHD prevention intravenously over four to six hours. Typical doses are in the range of 1.5-3.75 mg/kg. Infusions continue daily for one to two weeks. The drug remains active, targeting immune cells for days to weeks after treatment. This schedule is routinely adaptable for use in HIV treatment.

As can be seen, the T-cell depletors and modifiers can be used according to the invention in amounts and in administration regimens routinely determinable and analogous to known uses of such agents for other purposes.

In order to further strengthen the action of killing HIV cells, other drugs either alone or as mixtures of several drugs addressing different mechanisms, which are able to either kill HIV or inhibit HIV reproduction might be added to the regimen with the T-cell depletor or modifier. Today, HIV therapy normally consists of drug cocktails containing different types of drugs that attack at different stages of HIV proliferation. This therapy might be combined with anti-T-cell therapy to improve the efficacy of T-cell depletion or modification alone.

The treatment described above, consisting of T-cell depletion or modification with or without additional “conventional” anti-HIV therapy is administered until all viruses are eliminated. Thereafter, the immune system is allowed to recover. Since the system had been shut down in a controlled manner, any T-cells that are newly formed will be fully functional. Recovery of the immune system might be supported by drugs known in the art for this purpose. Examples are G-CSF or GM-CSF. However, any other applicable drugs or measures might as well be utilized.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of the applications, patents and publications, cited herein are incorporated by reference herein.

In more general terms, this invention relates to the treatment of viral infections that primarily proceed via the immune system by temporarily shutting down the system so that reproduction of the virus is no longer possible. Accordingly, the invention is not restricted to the treatment of HIV infection or AIDS. It may be used for any viral disease where it is possible to either temporarily reduce the functionality of host cells or to kill them and let them recover after substantially all viruses have been removed. It is understood that shutting down the host cells is a process that may significantly affect the functioning of the overall organism. Therefore, this invention is primarily recommended to be used in those cases where host cell manipulation can be handled appropriately. As detailed above, T-cells are a suitable target. Other examples would include B-cells, which are infected by, for example, the Epstein-Barr virus (EBV). Generally, the invention is applicable to any subcategory of such cells, including pre-B-cells, helper T-cells, cytotoxic T-cells, regulatory T-cells, etc. Further examples are T-cell subtypes such as macrophages that are infected by the Ebola, Marburg, Rubella or West Nile virus or by Leishmania, natural killer (NK) cells that are infected by HCMV, leukocytes that are infected by Mumps virus and monocytes infected by Lentiviruses. HTLV, human herpes virus and measles also infect the immune system targeting primarily T lymphocytes. The vaccinia virus infects primary hematolymphoid cells such as dendritic cells, monocytes and B-cells. Further examples are enteroviruses resulting in lymphocytic myocarditis, lymphocytic choriomeningitis virus and Coxsackie A-24 virus or other Coxsackie viruses, adenovirus 11 and 21 which lead to acute hemorrhagic conjunctivitis or cystitis, the Crimean-congo hemorrhagic fever virus, the virus leading to Dengue and dengue hemorrhagic fever virus, Hantavirus hemorrhagic fever virus, arenaviruses leading to, for example, Junin Argentinian hemorrhagic fever, Machupo Bolivian hemorrhagic fever or Lassa hemorrhagic fever, viruses targeting the erythroid progenitor cells such as parvoviruses, e.g. PV-B19 leading to Erythema infectiosum, the coronavirus resulting in SARS, and flaviviruses inducing yellow fever.

An example for an animal disease is Boma virus infection which primarily affects warm-blooded animals such as horses and sheep and which is believed to use hematogenous transmission. It has been reported that the foot and mouth disease virus is able to reproduce in macrophages or that macrophages could form a reservoir for the virus (Virology. 1995;207:503-9). Elimination of T-cells could thus destroy this potential reservoir and all viruses within them.

The host cells are killed or modified in function using any known method for the particular cells involved. The length of time involved is dependent on the cells and virus. Typically, the time period will be from a few (e.g., about 1-about 8 weeks) to a few months (e.g., about 2-about 12 months), depending also on the treatment modality used. The latter typically will involve antibodies to the host cells such as antibodies recognizing CD3 (e.g. T10B9), CD20 on B-cells (rituximab or ¹⁴C-ibritumomab tiuxetan), CD52 on B- and T-cells (alemtuzumab) or other epitopes found on the respective cell type but can also include available cytotoxic small molecules such as fludarabine and melphalan.

T-cell depletion, for example, can be achieved by a variety of methods. In addition to immunologic procedures which use T cell-specific antibody(ies) plus complement or toxin to kill the cells, T-cell depletion can also be achieved by physical methods such as separation by counterflow elutriation plus the Ceprate column or sheep cell resetting.

Such modalities are known for viral host cells. Typically, the degree of reduction of host cell number or functionality will be as close to zero as feasible, but reductions to 25% or lower, e.g., 15, 10, 5, 2, 1% etc are also useful. Typical functionalites to be reduced are those involved in viral reproduction at all stages.

This invention is directed towards use with any host cells. However, its preferred embodiment comprises host cells that are part of the immune system. Its most preferred embodiment comprises host cells that are part of the blood or lymphatic system.

### BRIEF DESCRIPTION OF DRAWINGS

FIG. 1 shown mean ALC as a function of time, and

FIG. 2 shown mean T-cell count as a function of time.

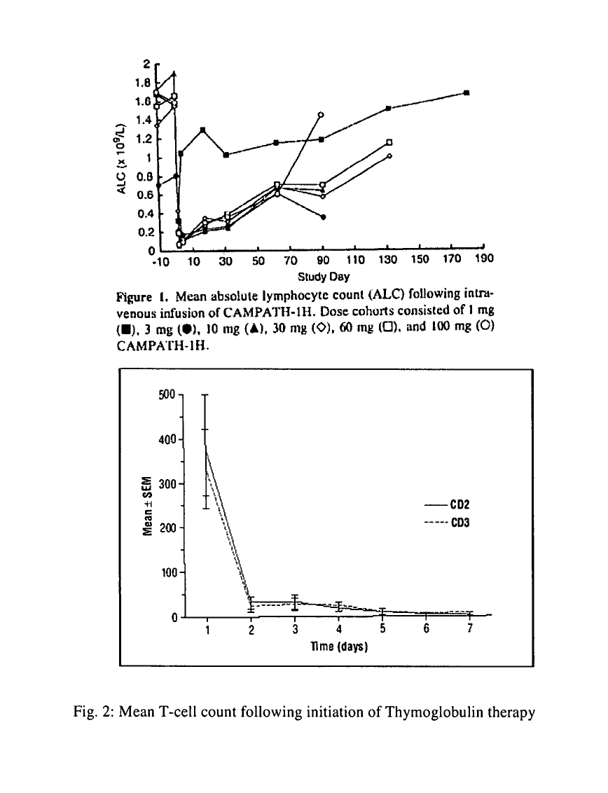

### EXAMPLES

### Example 1

A Phase II study for the treatment of HIV patients using a combination of alemtuzumab and Reverset.

### Study Design:

A total of 30 HIV-infected, treatment-naive individuals with CD4+ cell counts >50 cells/mm3 and plasma HIV-1 RNA levels >5,000 copies/ml are enrolled in a 10-day study. Subjects are randomized to one of two treatment arms, Reverset −200 mg once-a-day for 10 days, or Reverset −200 mg once-a-day for 10 days plus alemtuzumab every second day. The first dose of alemtzumab is 1 mg, the second dose 3 mg and the third dose is 5 mg. Any subsequent doses—if required by residual T-cell counts—are 5 mg. Alemtuzumab is infused IV over a period of 2 hours. Alternatively, alemtuzumab may be injected subcutaneously.

Study medication is administered in a double-blind fashion. Plasma samples are taken for HIV-1 RNA predose, on days 1, 2, 4, 8, 10 of treatment, and on days 11, 14, 21, 28 and 38 in the follow-up phase. Plasma samples for virus genotyping are taken at baseline, at the end of treatment, and at the follow-up visits.

### Example 2

A randomized, multicenter study compares the safety and efficacy of Lexiva plus ritonavir versus Kaletra (Lopinavir/ritonavir) over 48 weeks in ART (anti-retroviral therapy)-naive HIV-1 infected subjects while utilizing the Abacavir/lamivudine (ABC/3TC) FDC (fixed-dose combination tablet) as a NRTI (nucleoside reverse transcriptase inhibitor) backbone with or without adding alemtuzumab. This study evaluates the safety and efficacy of marketed HIV drugs [PI (protease inhibitor) plus NRTIs] given to HIV-infected patients who have not received prior therapy. All subjects will be screened and monitored at 12 scheduled clinic visits over a 48-week period. Abnormal laboratory values or certain side effects may require additional clinic visits over the course of the study. Alemtuzumab is added as an additional arm to either the Lexiva plus ritonar arm or to the Kaletra arm. A four-arm study is performed in which alemtuzumab is added to both the Lexiva plus ritonar arm and to the Kaletra arm. More details of original study (without the alemtuzumab arms) can be obtained from the NCI. The study no. is 100732, the NLM Identifier is NCT00085943 and the study is incorporated by reference herein. The dosing of alemtuzumab corresponds to the one described in Example 1.

### Study Design:

### Phase III, Treatment, Randomized, Open Label, Active Control, Parallel Assignment, Safety/Efficacy Study

### Patient Population:

Ages eligible for study: 18 years and above
Genders eligible for study: Both
Inclusion criteria:
Persons with HIV-1 infections who have not started any antiretroviral medication regimen HIV-1 RNA (viral load) >1,000 c/mL
Participants must be able to provide informed consent
Have not received more than 14 days of prior treatment with HIV drugs
Meet laboratory test criteria
Women must abstain from sexual intercourse or use acceptable contraception
Must be able to take study medications as directed and complete all study visits and evaluations during the 48-week study
   Exclusion criteria:
   Enrolled in other HIV treatment studies
   Pregnant or breastfeeding

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of U.S. Provisional Application Ser. No. 60/856,044, filed Nov. 2, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method of treating a viral infection comprising temporarily reducing the number or functionality of host cells for the virus or eliminating the host cells that are necessary for viral reproduction.

2. The method of claim 1, comprising temporarily reducing the number of host cells for the virus.

3. The method of claim 2 directed against a virus that causes immune suppression or that uses components of the immune system for their reproduction.

4. A method of treating West Nile virus infection comprising administering to a patient a T-cell depletor that effectively kills essentially all the patient's T-cells or a T-cell modifier that prevents West Nile virus from recognizing essentially all the T-cells.

5. A method of treating HTLV infection comprising administering to a patient a T-cell depletor that effectively kills essentially all the patient's T-cells or a T-cell modifier that prevents HTLV from recognizing essentially all the T-cells.

6. A method of treating EBV infection comprising administering to a patient a B-cell depletor that effectively kills essentially all the patient's B-cells or a B-cell modifier that prevents EBV from recognizing essentially all the B-cells.

7. The method of claim 2, comprising administering a monoclonal antibody directed against antigens present on the surfaces of components of the hematopoietic or immune system.

8. The method of claim 2, comprising administering a monoclonal antibody directed against CD3.

9. The method of claim 2, comprising administering a monoclonal antibody directed against CD4.

10. The method of claim 2, comprising administering a monoclonal antibody directed against CD20.

11. The method of claim 2, comprising administering a monoclonal antibody directed against CD52.

12. The method of claim 2, comprising administering muromonab-CD3.

13. The method of claim 2, comprising administering alemtuzumab.

14. The method of claim 2, comprising administering anti-thymocyte globulin.

15. The method of claim 2, comprising T-cell suicide gene transduction (Tk-gene).

16. The method of claim 2, comprising administering rituximab and/or ¹⁴C-ibritumomab tiuxetan.

17. The method of claim 7, comprising administering different monoclonal antibodies simultaneously.

18. The method of claim 2, comprising administering rituximab and alemtuzumab.

19. The method of claim 2, wherein a host cell depletor or host cell modifier is administered immediately after detection of viral infection.

20. The method of claim 2, wherein a host cell depletor or host cell modifier is administered until substantially no viruses are detectable.

21. The method of claim 5, wherein said T-cell depletor or T-cell modifier is administered immediately after detection of HTLV infection.

22. The method of claim 5, wherein said T-cell depletor or T-cell modifier is administered until substantially no HTLV viruses are detectable.

23. The method of claim 5, wherein said T-cell depletion or T-cell modification is started immediately after detection of HTLV infection and continued for about two years or a shorter period if substantially no HTLV viruses are detectable sooner.

24. The method of claim 2, wherein the treatment of viral infection is followed by treatment for strengthening of the immune system.

25. The method of claim 2, further comprising administering of a T-cell depletor or modifier in combination with or followed by G-CSF or GM-CSF treatment.

26. The method claim 5, comprising administration of a T-cell depletor in combination with conventional anti-HTLV therapy given either as monotherapy or as a drug cocktail.

27. The method of claim 5, comprising administration of a T-cell modifier in combination with conventional anti-HTLV therapy given either as monotherapy or as a drug cocktail.
